(19) United States
(12) Patent Application Publication
(10) Pub. No.: US 2013/0224203 A1
(43) Pub. Date: Aug. 29, 2013

(54) Pharmaceutical Composition of Recombinant Polyclonal Immunoglobulins

(71) Applicants: Tien-Li Lee, Millbrae, CA (US); Jane Wu Lee, Millbrae, CA (US); Gunnar Kaufmann, San Diego, CA (US); Henry Ji, San Diego, CA (US)

(72) Inventors: Tien-Li Lee, Millbrae, CA (US); Jane Wu Lee, Millbrae, CA (US); Gunnar Kaufmann, San Diego, CA (US); Henry Ji, San Diego, CA (US)

(73) Assignee: Sorrento Therapeutics, Inc., San Diego, CA (US)

(21) Appl. No.: 13/726,507

(22) Filed: Dec. 24, 2012

Related U.S. Application Data
(60) Provisional application No. 61/580,230, filed on Dec. 25, 2011.

(51) Int. Cl. A61K 39/395

(52) U.S. Cl. CPC A61K 39/395
USPC 424/135.1; 424/130.1; 435/69.6

(57) ABSTRACT

There is disclosed a recombinant pharmaceutical composition comprising a large plurality of recombinant immunoglobulins made by expression of a wide diversity of antibodies from a recombinant antibody library, preferably obtained from a wide diversity of human sources, synthetic or semi-synthetic germline immunoglobulin sequences, or a combination thereof, and then purified. Mammalian cell expression antibody libraries normally produce antibodies with significant diversity or different antibodies that bind to different targets. There is further disclosed a recombinant pharmaceutical composition made from a mammalian expression library configured to excrete, rather than display their antibodies on the cell surface, and then purified to form the pharmaceutical composition from the excreted antibodies.

### CROSS REFERENCE TO RELATED APPLICATION

This patent application claims priority to provisional patent application 61/580,230 filed 25 Dec. 2011.

### TECHNICAL FIELD

The present disclosure provides a recombinant pharmaceutical composition comprising a large plurality of recombinant immunoglobulins made by expression of a wide diversity of antibodies from a recombinant library, preferably obtained from a wide diversity of human sources, and then purified. Recombinant antibody libraries normally express antibodies with significant diversity or different antibodies that bind to different targets. The present disclosure provides a pharmaceutical composition made from a recombinant antibody library configured to excrete their antibodies, and then purified to form the pharmaceutical composition from the excreted antibodies.

### BACKGROUND

Intravenous immunoglobulins (IVIg), also known as intravenous immune globulins, are derived from the extracted plasma of many human donors (usually numbering over 1000) and pooled polyspecific immunoglobulins. IVIg is typically delivered intravenously to patients for a wide variety of disease treatments. Similar preparations are also administered subcutaneously for therapeutic indications. As described herein, “IVIg” shall refer to both IV and subcutaneous formulations. Given the relative proportion immunoglobulin classes or subtypes in human plasma during usual circumstances, most commercially available preparations are comprised primarily of IgG antibodies, with only minimal amounts of IgA and IgM.

Commercial preparations are currently available from a variety of companies, including CSL Behring, Baxter Healthcare Corp, Talecris Biotherapeutics, Octapharma, Bayer, Sandoz, Bio Products Laboratory, Instiuto Grifols SA, and others. Typical preparations are 5% to 20% IVIg in content with glycine, maltose, sucrose, sorbitol, or proline serving as stabilizers.

Human plasma derived immunoglobulin products were first used successfully in 1952 to treat immune deficiency via intramuscular injection. In 1981, intravenous immunoglobulin treatments subsequently were shown to also have efficacy in the treatment of autoimmune idiopathic thrombocytopenic purpura (ITP). Increasing utility for broader use has been demonstrated with time, and present clinical use falls within several general categories as follows:

1. Immune deficiencies such as severe combined immune-deficiencies, X-linked gammaglobulinemia, pediatric HIV, common variable immunodeficiency (CVID), hypogammaglobulinemia (primary immune deficiencies), Wiskott-Aldrich syndrome, acquired compromised immunity conditions (secondary immune deficiencies) featuring low antibody levels, and others.

2. Autoimmune diseases including ITP, Guillain-Barré syndrome, polymyositis, dermatomyositis, Wegener's granulomatosis, transplant/graft rejection (including allogeneic bone marrow and kidney transplant with ABO incompatibility), multiple sclerosis, myasthenia gravis, pemphigus, neonatal alloimmune thrombocytopenia, Churg-Strauss syndrome, chronic inflammatory demyelinating polyneuropathy (CIDP), and inflammatory diseases such as Kawasaki disease, and more.

3. Acute infections, covering a wide spectrum of conditions, including situations when the putative infective pathogen has not been yet identified in a patient.

4. Other categories, including CLL (chronic lymphocytic leukemia), multiple myeloma; additional experimental applications of IVIg include Alzheimer's Disease, infertility (usually due to spontaneous abortion), and many others.

The exact mechanisms of action through which IVIg confers its varying therapeutic effects for inflammatory and autoimmune conditions have not been completely defined;

however, it is likely due to multiple phenomena. Through its Fc regions, IVIg interacts with or blocks Fc receptors on macrophages leading to more limited phagocytosis action and reduced cell damage. IVIg interactions with B-cells, T-cells, and monocytes may also help regulate and modulate complement activation as well as induce self-tolerance. IVIg further decreases the level of cytokines and chemokines integral to the mediation of inflammatory responses. IVIg antibodies may also form immune complexes that then interact with Fc receptors on dendritic cells which in turn regulate and tamp down anti-inflammatory effects.

The polyclonal antibodies also neutralize and help remove abnormal host antibodies as well as pathogenic infective organisms. Another putative mechanism surmises that the magnitude of antibody challenge stimulates the patient's complement system, which then effectuates the removal of all antibodies, including the host-derived ones that cause autoimmune dysfunction. A recent study demonstrated IVIg applied to T cells led to reduced engagement of microglia and a resulting reduction of TNF-alpha and IL-10, which may partly explain how varying autoimmune diseases in the CNS may be treated with IVIg. IVIg also contains antibodies against granulocyte macrophage colony-stimulating factor, interferon, interleukin 1, and interleukin 6 which have debated, but potential independent or additive therapeutic significance.

The relative contribution of different mechanisms of action also varies with the primary disease entity or context in which IVIg may be used. For instance, with Kawasaki disease, it is believed that IVIg effectively binds activated complement components C3b and C4b, and thus inhibits the creation of the membrane attack complexes comprised of C5b-C9. In the case of treatment for patients with acquired hemophilia with anti-factor VIII antibodies, the abundance of anti-idiotype antibodies against autoantibodies results in neutralizing the disease inducing autoantibodies.

Additionally, donor derived IVIg contains proportions of IgG1-4 with trace amounts of IgA, and IgM, which relates closely to their relative representation in the serum of the human donors. However, this inflexible ratio does not allow for opportunities to optimize an immunoglobulin blend most appropriate for particular patients. For instance, although IgA only occurs in trace amounts in serum, it may represent around 75% of the total amount of antibodies produced in the body, underscoring its likely clinical importance. The main protective function of IgA is in the protection of mucosal tissues; therefore, IgA is typically found in the GI tract, respiratory epithelium, salivary glands, genitourinary tract, and eye. Administering human donor derived IVIg comprised mostly of IgG would not confer adequate protection for these regions in the context of a variety of diseases involving autoimmune, infectious, or inflammatory processes involving mucosal surfaces.

There are also some patients with auto-IgA sensitivity such that it would be preferred to have essentially no IgA (or even more minimal amounts of IgA) in an IVIg solution, so as not to trigger a sensitivity reaction.

And for patients with specific primary immune deficiencies involving the absence of a particular IgG subclass (for instance, IgG2 or IgG4), there are also no currently available options to selectively replace only the missing immunoglobulin fraction.

Despite its clinical utility in a vast array of important disease entities, broader use of IVIg is severely constrained by practical limitations of donor plasma supply and cost constraints due to the requirements of processing. Limitations of donor plasma supply is a particularly critical factor in many regions of the world with very low rates for voluntary blood donation, in which case net importation of IVIg is required in an attempt to meet clinical demand and need. Due to such shortages worldwide, many healthcare institutions ration the use of IVIg, even when full reimbursement is available, given the desire to allocate use only for those patients with the most serious conditions, although many more patients could benefit from wider availability.

There are also quality control challenges for human donor derived IVIg. Despite careful processing, there remains a measurable risk of viral (Hepatitis B, C, HIV, and others) or Creutzfeldt-Jacob disease contamination. Subjective fear of such risks by patients also affects adoption of IVIg treatment, even when clinically indicated and available. Donor derived IVIg also may contain trace amounts of cytokines, soluble CD4, CD8, and HLA molecules which may negatively affect clinical outcomes depending on the indication for use. And given the variable donor pool, it is impossible to maintain optimal batch-to-batch consistency with donor derived IVIg.

Therefore, there is a need in the art to obtain IVIg pharmaceutical compositions by recombinant protein means and avoid the aforementioned risks to obtain such pharmaceutical compositions by purification from human plasma sources. The present disclosure provides a significant solution to this problem.

### SUMMARY

The present disclosure provides a pharmaceutical composition of polyclonal fully human antibodies selected from the group consisting of a plurality of IgG1, IgG2, IgG3 and IgG4 antibodies, IgM antibodies, IgA antibodies, IgE antibodies, IgD antibodies, single chain scFv antibodies, Fab antibody fragments, domain antibodies of homer-dimer of heavy chains or light chains of the antibodies, antibodies of non-immunoglobulin scaffolds comprising a functional variable domain sequence of a heavy and/or a light chain of antibodies, and combinations thereof. Preferably, the pharmaceutical composition comprises at least about 100 different antibodies, wherein the differences between the antibodies are the sequences of their variable domain regions in a heavy chain and a light chain. Preferably, the pharmaceutical composition has at least 100 different binding specificities.

The present disclosure provides a cell based method to manufacture, by recombinant means, fully human polyclonal antibody formulations comprised of varying proportions of antibody classes and subclasses. Preferably, the antibody classes are selected from the group consisting of IgG antibodies, Ig antibodies, IgA antibodies, IgM antibodies, Ig-like antibodies, scFv single chain antibodies, scFv-Fc antibodies, Fab antibody fragments, and combinations thereof.

The present disclosure further provides a recombinant pharmaceutical composition comprising at least about 100 different secreted recombinant polypeptides selected from the group consisting of immunoglobulins, Ig-like antibodies, Fab fragments, scFv antibodies and combinations thereof. Preferably, the pharmaceutical composition comprises at least about 1000 different secreted recombinant polypeptides. Preferably, the pharmaceutical composition comprises at least about 10⁴ different secreted recombinant polypeptides. Preferably, the differences between the antibodies are the sequences of their variable domain regions in a heavy chain and a light chain. Preferably, the differences between the antibodies are the sequences of their variable domain regions in a heavy chain and a light chain. Preferably, the antibodies are in a form selected from the group consisting of IgG1, IgG2, IgG3, IgG4, IgA1, IgA2, IgM, IgD, rIVIG, IgE, and combination thereof.

The present disclosure further provides a cell based method to manufacture, by recombinant means, fully human polyclonal antibody formulations comprising varying proportions of antibody classes and subclasses. Preferably, the antibody classes are selected from the group consisting of IgG antibodies, Ig antibodies, IgA antibodies, scFv single chain antibodies, scFc-Fc antibodies, Fab antibody fragments, and combinations thereof.

The present disclosure further provides a method for treating a disease involving mucosal tissue, lung tissue or eye tissue, comprising administering an effective amount of a recombinant pharmaceutical composition comprising at least about 100 different secreted recombinant polypeptides selected from the group consisting of immunoglobulins, Fab fragments, scFv-Fc antibodies, scFv antibodies and combinations thereof. Preferably, the pharmaceutical composition comprises at least about 1000 different secreted recombinant polypeptides. Preferably, the pharmaceutical composition comprises at least about 10⁴ different secreted recombinant polypeptides. Preferably, the recombinant pharmaceutical composition contains primarily polyclonal IgA class antibodies.

### BRIEF DESCRIPTION OF THE DRAWINGS

FIG. 1 shows a vector named as pIgH (FIG. 1), which comprises a mammalian episomal origin of replication (such as SV40 ori), an antibiotic resistance marker for antibiotic selection (such as neomycin gene NeoR), and a plasmid origin of replication. The pIgH comprises also a promoter for driven gene expression in mammalian cells (such as CMV promoter), which drives the down-stream full-length immunoglobulin heavy chain gene expression. The pIgH comprises also a constant region (CH) sequence of the heavy chain, such as IgA₁, IgA₂, IgD, IgE, IgG₁, IgG₂, IgG₃, IgG₄, and IgM. The variable domain sequences of the heavy chain of the immunoglobulin gene (VH) or VH gene library (VH Library) inserts will be derived from a human antibody library and are inserted recombinantly into the insertion sites as designed in the pIgH vector to generate a FL immunoglobulin heavy chain library (IgH Library).

FIG. 2 shows the composition of vector pIgL which comprises a mammalian episomal origin of replication (such as SV40 ori), an antibiotic resistance marker for antibiotic selection (such as neomycin gene NeoR), and a plasmid origin of replication. The pIgL comprises also a promoter for driven gene expression in mammalian cells (such as CMV promoter), which drives the down-stream full-length immunoglobulin light chain gene expression. The pIgL comprises a variable domain and a constant region (CL) sequence of the light chain, such as kappa (k) or lambda (l). A library of FL light chains derived from a human antibody library is inserted into pIgL vector to generate a FL immunoglobulin light chain library (IgL Library). FIG. 3 shows the composition of vector pIgH&L which comprises a mammalian episomal origin of replication (such as SV40 ori), an antibiotic resistance marker for antibiotic selection (such as neomycin gene NeoR), and a plasmid origin of replication. The pIgH&L comprises also a promoter for driven gene expression in mammalian cells (such as CMV promoter), which drives the down-stream full-length immunoglobulin heavy and light chain gene co-expression. The heavy and light chain co-expression is achievable by an internal ribosomal entry site (IRES) linked in between the FL H and L chains.

FIG. 4 shows the composition of vector pscFv-Fc which comprises a mammalian episomal origin of replication (such as SV40 ori), an antibiotic resistance marker for antibiotic selection (such as neomycin gene NeoR), and a plasmid origin of replication. The pscFv-Fc comprises also a promoter for driven gene expression in mammalian cells (such as CMV promoter), which drives the down-stream full-length immunoglobulin heavy chain gene expression. The pscFv-Fc comprises also a constant region (CH) sequence of the heavy chain, such as IgA₁, IgA₂, IgD, IgE, IgG₁, IgG₂, IgG₃, IgG₄, and IgM.

### DETAILED DESCRIPTION

The present disclosure provides cell based methods to manufacture, by recombinant means, fully human polyclonal antibody formulations comprised of varying proportions of antibody classes and subclasses. For example, a formulation with a greatly increased proportion of IgA antibodies (including dimeric or oligomeric IgA1 and IgA2 optionally including J-chain and secretory components) relative to IgG, is for diseases with infectious, autoimmune, inflammatory, or other pathology at mucosal surfaces. For patients that might have IgA hypersensitivity, a formulation comprised mostly of IgG antibodies with the near complete absence of IgA would be provided in that setting. Other formulation mixes are also anticipated herein for different therapeutic indications. Since it is possible to selectively manufacture immunoglobulins of a particular class, controlling the proportion of IgG, IgA, IgM, and others (along with subclasses) is not dependent on filtration or negative selection means, as would need to be performed with donor derived immunoglobulins from plasma.

In one example, IgA, such as dimeric or oligomeric IgA complexed with a J-chain and a secretory component, would be able to transport from an intravenous or subcutaneous route to various mucosal tissues via active transport mechanisms. This is made possible partly by utilizing the natural role of the polymeric Ig receptor (pIgR) found throughout the human body. Such target tissues for the manufactured IgA could include respiratory epithelium, the GI tract, the eye, the genitourinary system, mouth, and nasal cavities.

Alternatively, the IgA formulations may be delivered in manners other than IV or SC, for instance, via an oral, nasal, or genitourinary (catheter), pulmonary, eye (drops or intraocular injection), or rectal route for direct delivery to mediate infectious, auto-immune, or inflammatory disease processes in those very tissues. IgA, especially dimer and oligomeric IgA, are designed to resist degradation in those environments, and may thus be more effective in addressing local mucosal pathology better than any other immunoglobulin classes. IgA plays a significant role in the therapeutic benefit conferred when human infants ingest antibody rich colostrum from breast milk. Pulmonary delivery via a mist or solid particle inhalation may be able to deliver therapeutic antibodies to both upper and lower respiratory tract targets; currently, IVIg is generally not effective in addressing upper respiratory tract conditions. IgA rich IVIg may also facilitate mediation of Graves' ophthalmopathy, since human donor derived IVIg does not confer significant therapeutic effects in the eye. About 75% of total body antibody production is in the IgA subclass despite its poor representation in plasma, so its role in disease cannot be understated.

These aforementioned manufactured human antibodies would be all derived from a gene library of variable domains of immunoglobulin heavy and/or light chains sufficiently containing a vast variety of the immunological gene repertoire, then subjected to expression and manufacturing (via CHO cell, E. coli, yeast, plant, algae, or other means) designed or enhance or to retain the wide spectrum of diversity within the library used. The goal would be the ability to create a polyclonal antibody formulation that achieves equivalence or superiority for therapeutic efficacy as compared with human donor derived IVIg. Even in the circumstance that such manufactured IVIg is less efficacious than donor derived IVIg, there would still be broad clinical utility, given the extremely limited supply and current rationing of donor-derived IVIg. Consistency would also be more easily maintained than with donor IVIg, where batch-to-batch variability is a significant problem; the spectrum of human donor's changes with each processing run for donor plasma derived IVIg products.

With a cell based manufacturing system, viral contamination as well as Creutzfeldt-Jacob disease no longer are realistic concerns, as they still are with human donor sourced product. Trace amounts of cytokines, soluble CD4, CD8, and HLA molecules also no longer interfere with the purity of the intended product. Scalability is also a significant advantage, as supply constraints for the human donated plasma will always limit traditional IVIg production, drive cost, and limit supply to those patients who would otherwise derive significant benefit. Additionally, the manufacturing cost to engineer antibodies has been rapidly decreasing with new technological and process improvements over time, such that manufacturing polyclonal IVIg should progressively become ever more cost-efficient to produce in the future.

Additionally, a further enhancement to the strategy to manufacture IVIg may be to generate or collect a gene library from human patient populations that have already demonstrated rare but beneficial and successful humoral immunity in the context of various diseases. As one example, a minority of patients exposed to Hepatitis C are able to spontaneously clear the virus, thus rendering a cure. Ostensibly, passive immunity from IVIg derived from clones of immunoglobulin cells from those individuals may help confer the ability to clear the virus as well.

Another proposed use for manufactured IVIg, including IVIg derived from libraries of patients that have successfully cleared certain viral infections, is chronic administration or closely repeated administration with the aim to facilitate viral clearance for a patient.

Intracellular antibody-mediated degradation (IAMD) is a natural process whereby virions are first bound by IgG antibodies extracellularly. As the IgG bound virion infects host cells, TRIM21 in cytosol binds to the IgG-virion complex and then becomes conjugated to ubiquitin, which in turn directs the virion and antibody elements to proteasomes for degradation. This process has been demonstrated for adenovirus but is presumed to be likely effective at least against other non-enveloped viruses and possibly against a wide range of viruses. The intracellular process utilizing TRIM21 is expressed in most human tissues, is highly conserved, and is highly resistant to evasion via mutation by the pathogen, since the intracellular interaction with TRIM21 is via the IgG complex. Even if virions develop resistance to ubiquitination, it is not likely to evade this intracellular process, since TRIM21 is auto-ubiquitinated without direct interaction with the virion.

The strategy of long-term or repeat administration of manufactured IVIg to clear viral infections should be clinically effective due to other factors as well. The exogenous introduction of a broad spectrum of polyclonal antibodies would diminish virion escape through mutagenesis and evolution. This is because there is high likelihood that there are specific antibody binders in the manufactured IVIg pool with avidity to (essentially “anticipating”) newly mutated antigens, thus preventing rapid virion replication with escape mutants. This effect may be further enhanced by creating manufactured IVIg using cell line samples from patients that have demonstrated rare, robust, and successful clearance of the particular virion of interest. A further effect in selecting such specific cell lines, is that the required total amount of manufactured IVIg required for treatment may be dramatically less, which would diminish hospitalization time required for infusion (currently requiring up to 4 days for traditional donor based IVIg), complications from volume and osmotic effects, hypersensitivity reactions, and off target effects; reduced required manufactured IVIg would also significantly lower production costs.

Yet another proposed refinement for the utilization of manufactured IVIg, to provide advantages not easily replicated using donor plasma as a starting source, is to greatly increase the percentage of certain antibody subclasses. One specific strategy is to increase the population of IgG3 subclass antibodies. In commercially available IVIg preparations available today, IgG1 and IgG2 comprise approximately 90% or more of the total antibody population, with IgG3 only comprising 2-7%. However, among IgG subclasses, IgG3 generally is the most potent complement activator and has high affinity with the Fc receptor on phagocytic cells.

Naturally circulating antibodies or immunoglobulins are produced by different B cells with each individual B cell producing immunoglobulins with one specific structure. The natural structure of immunoglobulins (Igs) is a four-polypeptide chain construct and structure comprised of two identical heavy (H) chains (about 450-600 amino acids) and two identical light (L) chains (about 230 amino acids). Different antibody classes are defined by the H chains and classified into the five major classes or isotypes: IgA, IgD, IgE, IgG, and IgM. The IgG class can be further divided into subclasses including IgG1, IgG2, IgG3 and IgG4. The IgA class can be further subdivided into subclasses IgA1 and IgA2. There are two different L chains: the lambda (λ) chain and the kappa (κ) chain.

The specificity and affinity of a particular immunoglobulin molecule for a selected antigen is determined primarily by the highly variable N-terminal regions of the H and L chains: the variable (V) domains. The V domain is followed by several constant domains in the H chain and one constant (C) domain in the L chain. The part of the immunoglobulin that binds to an antigen is defined as antigen binding fragment (the “Fab”). The Fab is composed of an H chain C and V domain (VH-CH), and an L chain C and V domain (VL-CL). Another part of the immunoglobulin is termed as the crystallizable Fc region or “tail” end of the antibody. The Fc region is composed of the constant regions of two heavy chains. The CH and CL domains of the immunoglobulins are not directly involved in specific antigen binding. VH and VL regions directly bind and determine the specificity and affinity of the antigen binding. The V regions of the H and L chain are further comprised of four relatively conserved framework segments or framework regions (FR) and in between the FR regions are three hypervariable complementarity determining regions (CDRs). The CDRs physically bind to the antigen and determine the specificity and affinity of the antigen binding to a particular antigen epitope.

The immunoglobulin H chain's V regions are determined by the V genes, the diversity (D) genes, and the joining (J) genes. Recombination of different V-D-J domains give rise to a large diversity in the V regions of immunoglobulin genes. The L chain lacks the D gene; thus, the L chains are comprised of VJ gene segments. The different combinations of H chains and L chains produce the vast diversity of the immunoglobulin repertoire: a) the IVIg when isolated from the natural peripheral mononuclear blood cells (PMBC) of donors and b) the antibody library Ig (ALIG) when produced in an in vitro expression system.

An antibody library is generally constructed by generating the VH and VL gene repertoires of immunoglobulin genes. VH and VL can also be constructed in Fab format or in single chain antibody (SCA) or in single chain fragment variable (scFv) format. Fab or scFv can be cloned into expression vectors and expressed onto the surface of filamentous bacteriophage to form the phage display antibody library or onto mammalian or yeast surfaces (yeast displayed antibody library).

Winter et al (U.S. Pat. No. 6,291,158) and Lerner et al (U.S. Pat. No. 6,291,161) described an original scheme in constructing an antibody library. The diversity and size of a typical human antibody library is estimated to be on the order of 10⁶ to 10¹⁰ different antigen specificities, while a typical person carries in the blood circulation on the order of 10⁷ to 10⁸ different antigen specificities. Thus, the antibody specificities produced from a human antibody library is on par or greater than the ones from the pooling of the IVIg from individuals.

Expression Libraries

Specialized human antibody libraries are produced from PMBCs of particular patient populations such as cancer patients, patients infected by certain pathogens, or patients with autoimmune diseases. The resulting antibody library contains human antibodies with very high avidity and specificity for particular diseases.

The antibody libraries in the scFv, Fab, scFv-Fc or full Ig formats produce vast immunoglobulin specificities as scFv, Fab, scFv-Fc or Ig antibodies. In the circumstance that libraries are in the form of full Ig, the Fc portion are engineered to be different Ig classes, i.e. IgG (including IgG1-4), IgA (including IgA1-2), IgM, IgD and IgE. For example, an IgA antibody library is produced when the V regions of the H chain are linked to a common IgA Fc sequence. The antibodies produced from the IgA antibody library are all of IgA form. An IgM antibody library represents the truly naïve antibody library, which usually bears only the immunoglobulins prior to any antigen exposure.

A human antibody library is also generated through synthetic or semi-synthetic assembly of the VDJ sequences for the H chain and the VJ sequences for the L chain. The V, D, and J sequences of the H chain VDJ assembly and the V and J sequences of the L chain VJ assembly can be derived from the germline V, D, and J sequences widely available in germline immunoglobulin sequence databases. The germline immunoglobulin library generated from synthetic germline variable domain sequences offers a universal germline recombinant IVIg as the germline sequences are finite and consensus for all human being. The manufactured recombinant germline IVIg are independent from individuals, thus, providing uniformed and consistent manufactured germline IVIg. Semi-synthetic antibody libraries can be generated when certain preferred FR sequences are used and random CDR sequences engineered into CDRs, especially the most diverse CDR3 region. Again the manufactured semi-synthetic IVIg offer consistency and uniformity that is independent from individual donors.

Vast and diverse human antibodies are produced from the diverse and various human antibody libraries. The antibody library constructed in various ways: a) in a phage expression vector that can produce scFv antibodies; b) in a bacterial expression vector that can produce Fab, scFv and scFv-Fc antibodies; and c) in a mammalian expression vector or vectors can produce Fab, scFv-Fc or full Ig antibodies, typically expressed in Chinese hamster ovary (CHO) cells or human embryonic kidney (HEK) 293 cells.

These expressed antibodies of human ALIGs (hALIGs) are routinely isolated and purified. The diversity of immunoglobulin specificity of the purified hALIGs are on par or exceeding that of the IVIGs, thus, the hALIGs can be a replacement for the IVIGs for many of the aforementioned different clinical utilities.

Manufacturing the IVIg Recombinant Composition

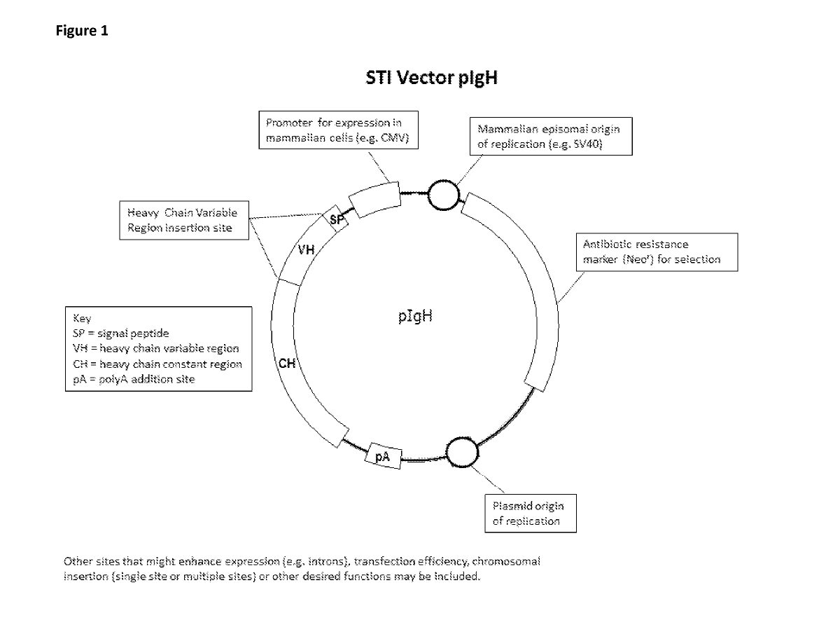

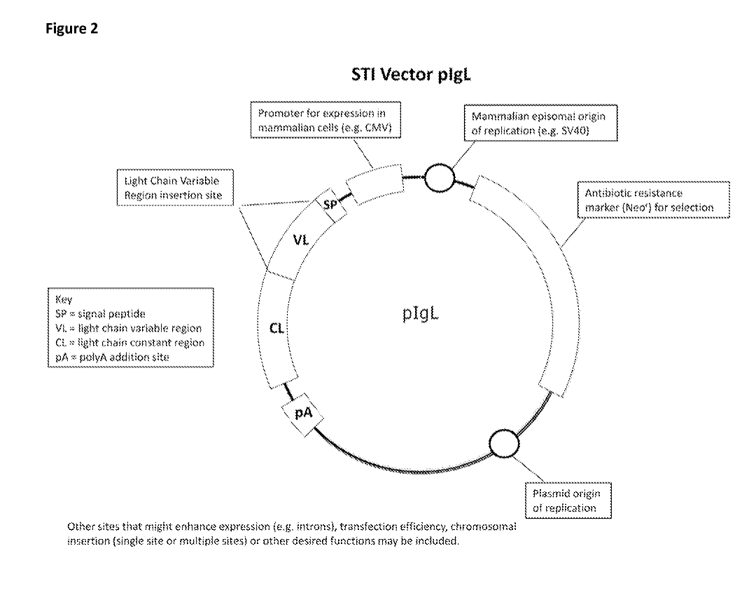

A vector named pIgH (FIG. 1), which comprises a mammalian episomal origin of replication (such as SV40 ori), an antibiotic resistance marker for antibiotic selection (such as neomycin gene NeoR), and a plasmid origin of replication can be used as a highly diverse antibody source. The pIgH comprises also a promoter for driven gene expression in mammalian cells (such as CMV promoter), which drives the down-stream full-length immunoglobulin heavy chain gene expression. The pIgH comprises also a constant region (CH) sequence of the heavy chain, such as IgA₁, IgA₂, IgD, IgE, IgG₁, IgG₂, IgG₃, IgG₄, and IgM . The variable domain sequences of the heavy chain of the immunoglobulin gene (VH) or VH gene library (VH Library) inserts are derived from a human antibody library and are inserted recombinantly into the insertion sites as designed in the pIgH vector to generate a FL immunoglobulin heavy chain library (IgH Library).

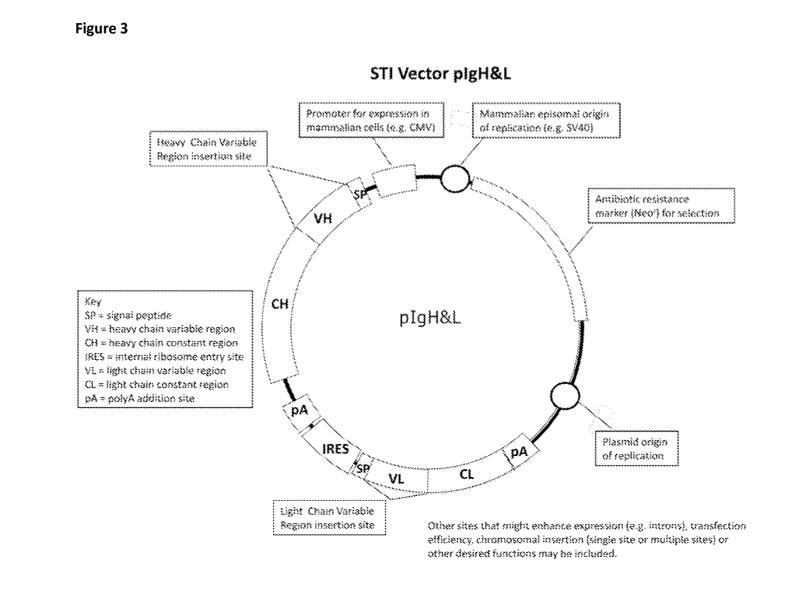

In another embodiment, the vector named as pIgL (FIG. 2), which comprises a mammalian episomal origin of replication (such as SV40 ori), an antibiotic resistance marker for antibiotic selection (such as neomycin gene NeoR), and a plasmid origin of replication. The pIgL comprises also a promoter for driven gene expression in mammalian cells (such as CMV promoter), which drives the down-stream full-length immunoglobulin light chain gene expression. The pIgL comprises a variable domain and a constant region (CL) sequence of the light chain, such as kappa (k) or lambda (l). A library of FL light chains derived from a human antibody library is inserted into pIgL vector to generate a FL immunoglobulin light chain library (IgL Library).

In a preferred embodiment, a FL IgH Library and a FL IgL library are co-transfected into a mammalian cell culture and FL IgH and FL IgL genes are co-expressed in individual co-transfected mammalian cells and secreted into the culture medium as full length human immunoglobulins. A cell culture of 10⁶ to 10¹⁰ cells in a laboratory setting expresses and secretes up to 1 g per liter of fully assembled FL human immunoglobulins into the culture medium. The cell culture in a good manufacturing practice (cGMP) environment exceeds 10¹⁰ cells, thus, produce scalable recombinant IVIgs for clinical use.

In one preferred embodiment, a single FL heavy chain immunoglobulin (sIgH) or a library of FL heavy chain immunoglobulins (IgH Library) is transfected into mammalian cells.

The transfected mammalian cells are made permanent by antibiotics selection (such as G418 drug selection) when neomycin resistance gene is expressed. The permanent mammalian cells expressing one or a plural of IgH are termed an IgH-Expressing Line.

In another preferred embodiment, a single FL light chain immunoglobulin (sIgL) or a library of FL light chain immunoglobulin (IgL Library) are transfected into mammalian cells. The transfected mammalian cells are made permanent by antibiotics selection (such as G418 drug selection) when neomycin resistance gene is expressed. The permanent mammalian cells expressing one or a plural of IgL are termed an IgL-Expressing Line.

In another preferred embodiment, an IgH Library is transfected into an IgL-Expressing Line so that the transfect cells of IgL-Expressing Line express fully assembled immunoglobulins in the transfected IgL-Expressing Line cells, with each comprising hundreds (10²) to hundreds thousands (10⁵) IgHs and a single IgL. A cell culture of 10⁶ to 10¹⁰ cells may produce and secrete up to 1 g per liter of fully assembled FL human immunoglobulins into the culture medium. The cell culture in a good manufacturing practice (cGMP) environment exceeds 10¹⁰ cells, thus, produce scalable recombinant IVIgs for clinical use.

In another preferred embodiment, an IgL Library is transfected into an IgH-Expressing Line so that the transfect cells of IgH-Expressing Line express fully assembled immunoglobulins in the transfected IgH-Expressing Line cells, with each comprising hundreds (10²) to hundreds of thousands (10⁵) of IgLs and a single IgH. A cell culture of 10⁶ to 10¹⁰ cells produce and secrete 5 mg/L to 10 g/L, preferably 50 mg/L to 1 g/L, more preferably 50 mg/L to 200 mg/L for transient expression, or up to 1 g per liter of fully assembled FL human immunoglobulins into the culture medium. The cell culture in a good manufacturing practice (cGMP) environment exceeds 10¹⁰ cells, thus, produce scalable recombinant IVIgs for clinical use.

In another embodiment, the pIgH and pIgL vectors, of different backbones (such as they contain different antibiotics resistant genes), are used to construct both full-length heavy and light chain immunoglobulin libraries. The heavy and light chain immunoglobulin libraries are constructed initially by transforming the pIgH and pIgL constructs in prokaryotic cells and the isolated vectors comprising either heavy or light chain immunoglobulin genes in plasmids form. The pIgH and pIgL are co-transfected into a mammalian cell for co-expression of multiple different types of heavy chains and light chains in each individual mammalian cell. The cells express and secrete properly configured and assembled immunoglobulins into the cell culture media.

In one embodiment, the vector named as pIgH&L (FIG. 3), comprises a mammalian episomal origin of replication (such as SV40 ori), an antibiotic resistance marker for antibiotic selection (such as neomycin gene NeoR), and a plasmid origin of replication. The pIgH&L vector also comprises promoter for driven gene expression in mammalian cells (such as CMV promoter), which drives the down-stream full-length immunoglobulin heavy and light chain gene co-expression. The heavy and light chain co-expression is achieved by an internal ribosomal entry site (IRES) linked in between the FL H and L chains.

In a preferred embodiment, a library of variable domain sequences of the heavy chain are inserted into the VH insertion site of pIgH&L to form an IgH Library. A single common FL light chain is inserted in the vector pIgH&L, which upon transfection into a mammalian cell culture, co-expresses a library of FL IgH and a single common FL sIgL gene in individual transfected mammalian cells. The antibodies are secreted into the cell culture media. Each individual cell expresses hundreds (10²) to hundreds of thousands (10⁵) of fully assembled FL immunoglobulins. All of the FL immunoglobulins in each of the mammalian cells comprise a single common FL light chain (sIgL) and different FL IgH chain of fully assembled immunoglobulins. A cell culture of 10⁶ to 10¹⁰ cells expresses and secretes 5 mg/L to 10 g/L, preferably 50 mg/L to 1 g/L, more preferably 50 mg/L to 200 mg/L for transient expression, or up to 1 g per liter of fully assembled FL human immunoglobulins., all comprising a sIgL, into the cell culture media.

In another preferred embodiment, a library of FL light chains is inserted into the light chain insertion site of pIgH&L to form an IgL Library. A single common FL heavy chain is inserted in the vector pIgH&L, which upon transfection into a mammalian cell culture co-expresses a library of FL IgL and a single common FL sIgH genes in individual transfected mammalian cells. Each individual cell expresses hundreds (10²) to hundreds thousands (10⁵) of fully assembled FL immunoglobulins. The FL immunoglobulins in each of the mammalian cells comprise a single common FL heavy chain (sIgH) and different FL IgL chains of fully assembled immunoglobulins. A cell culture of 10⁶ to 10¹⁰ cells expresses and secretes 5 mg/L to 10 g/L, preferably 50 mg/L to 1 g/L, more preferably 50 mg/L to 200 mg/L for transient expression, or up to 1 g per liter of fully assembled FL human immunoglobulins into the culture medium.

In another preferred embodiment, the pLentiIgH+L vector is part of a lentivirus-based expression plasmid system, in which case pLentilgH+L is the “transfer vector plasmid” containing cis-acting genetic sequences necessary for the vector to infect the target cells, a packing signal, and restriction sites for the transfer of the IgH+L library into the target cells. The pLentiIgH+L vector can either be used for transfection of mammalian cells, such as HEK293 cells, or used in combination with 1 or 2 packing plasmids encoding lentiviral structural proteins. The lentiviral structural proteins are required for generation of infective lentiviral particles containing an IgH+L library that will, in turn, be used to transfect mammalian cells, such as HEK293 cells, for co-expression of multiple different types of heavy chains and light chains in each individual mammalian cell. The cells express and secrete properly configured and assembled immunoglobulins into the cell culture media. A cell culture of 10⁶ to 10¹⁰ cells expresses and secretes 5 mg/L to 10 g/L, preferably 50 mg/L to 1 g/L, more preferably 50 mg/L to 200 mg/L for transient expression, or up to 1 g per liter of fully assembled FL human immunoglobulins into the culture medium. The cell culture in a good manufacturing practice (cGMP) environment exceeds 10¹⁰ cells, thus, produce scalable recombinant IVIgs for clinical use.

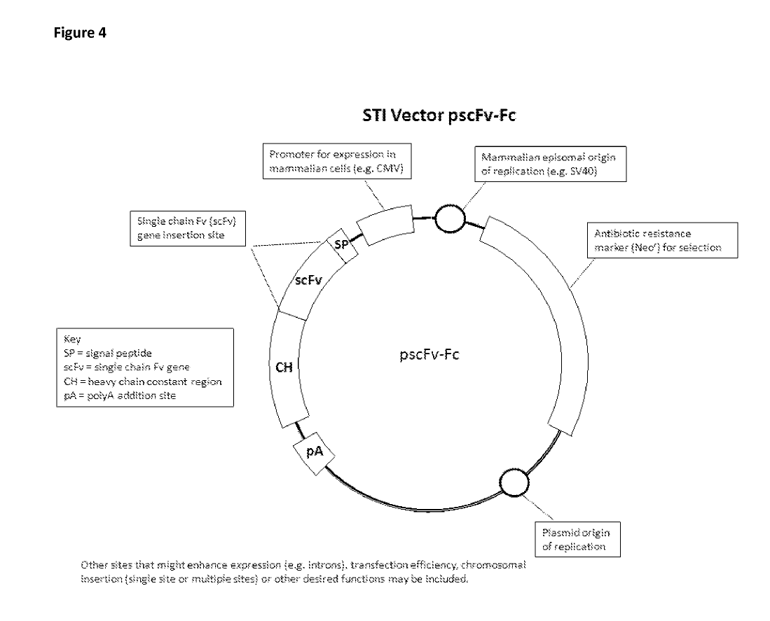

In one embodiment, the vector named as pscFv-Fc (FIG. 4), comprises a mammalian episomal origin of replication (such as SV40 ori), an antibiotic resistance marker for antibiotic selection (such as neomycin gene NeoR), and a plasmid origin of replication. The pscFv-Fc comprises also a promoter for driven gene expression in mammalian cells (such as CMV promoter), which drives the down-stream full-length immunoglobulin heavy chain gene expression. The pscFv-Fc comprises also a constant region (CH) sequence of the heavy chain, such as IgA₁, IgA₂, IgD, IgE, IgG₁, IgG₂, IgG₃, IgG₄, and IgM. The variable domain sequences of the heavy chain (VH) and the light chain (VL) of the immunoglobulin inserts are derived, for example, from a mammalian human antibody library as single chain Fv antibody fragments, such as genes encoding VH and VL (either in VH-VL or VL-VH orientation) connected via a peptide linker, and are inserted recombinantly into the insertion sites as designed in a pscFv-Fc vector to generate a immunoglobulin-like scFv-Fc library (scFv-Fc Library). The cell culture in a good manufacturing practice (cGMP) environment exceeds 10¹⁰ cells, thus, produce scalable recombinant IVIgs for clinical use.

In a preferred embodiment, a scFc-Fc Library is transfected into a mammalian cell culture and scFv-Fc genes are expressed in individual transfected mammalian cells and secreted into the culture medium as immunoglobulin-like human scFv-Fc molecules. A cell culture of 10⁶ to 10¹⁰ cells potentially expresses and secretes 10⁸ to 10¹⁵ different fully assembled immunoglobulin-like human scFv-Fc molecules into the culture medium with yields of 5 mg/L to 10 g/L, preferably 50 mg/L to 1 g/L, more preferably 50 mg/L to 200 mg/L for transient expression, or up to 1 g per liter. The cell culture in a good manufacturing practice (cGMP) environment exceeds 10¹⁰ cells, thus, produce scalable recombinant IVIgs for clinical use.

In another embodiment, a scFc-Fc Library is transfected into a prokaryotic host cell culture, such as Escherichia coli or Bacillus subtillis, and scFv-Fc genes are expressed in individual transfected prokaryotic cells and secreted into the culture medium as immunoglobulin-like human scFv-Fc molecules. A cell culture of 10¹² to 10¹⁵ cells expresses and secretes 5 mg/L to 10 g/L, preferably 50 mg/L to 1 g/L, more preferably 50 mg/L to 200 mg/L for transient expression, or up to 1 g per liter of immunoglobulin-like human scFv-Fc molecules into the culture medium. The cell culture in a good manufacturing practice (cGMP) environment exceeds 10¹⁰ cells, thus, produce scalable recombinant IVIgs for clinical use.

In one preferred embodiment, a scFc-Fc Library is transfected into a eukaryotic host cell culture, such as algae, e.g. Chlamydomonas reinhardtii or Phaeodactylum tricornutum, tobacco, e.g. Nicotiana tabacum L, or rice, e.g. Oryza sativa, and scFv-Fc genes are expressed in individual transfected plant cells and secreted into the culture medium as immunoglobulin-like human scFv-Fc molecules. A cell culture of 10⁶ to 10¹⁰ cells potentially expresses and secretes 10⁸ to 10¹⁵ different fully assembled immunoglobulin-like human scFv-Fc molecules into the culture medium 5 mg/L to 10 g/L, preferably 50 mg/L to 1 g/L, more preferably 50 mg/L to 200 mg/L for transient expression, or with yields up to 1 g per liter. Alternatively, the scFv-Fc molecules can be recovered from the biomass of the plant cells 5 mg/L to 10 g/L, preferably 50 mg/L to 1 g/L, more preferably 50 mg/L to 200 mg/L for transient expression, or with yields up to 1 g per kilogram of biomass. The cell culture in a good manufacturing practice (cGMP) environment exceeds 10¹⁰ cells, thus, produce scalable recombinant IVIgs for clinical use.

In another embodiment, a scFv-Fc Library is transfected into a eukaryotic host cell culture, such as insect cells using a baculovirus-based transfection system, and scFv-Fc genes are expressed in individual transfected eukaryotic cells and secreted into the culture medium as immunoglobulin-like human scFv-Fc molecules. A cell culture of 10⁶ to 10¹⁰ cells potentially expresses and secretes 10⁸ to 10¹⁵ different fully immunoglobulin-like human scFv-Fc molecules into the culture medium 5 mg/L to 10 g/L, preferably 50 mg/L to 1 g/L, more preferably 50 mg/L to 200 mg/L for transient expression, or with yields up to 1 g per liter. The cell culture in a good manufacturing practice (cGMP) environment exceeds 10¹⁰ cells, thus, produce scalable recombinant IVIgs for clinical use.

### Example 1

This example illustrates the manufacture of the disclosed recombinant IVIg pharmaceutical composition. A 25L cell culture wave bag is seeded with CHO-S cells and used for run volumes from 10L to 25L. To begin a run the wave bag is attached to a wave bioreactor rocking platform and the CO₂Mix20 controller set at 5% CO₂. The platform is set to maintain the bag at 37° C. to begin the run. The bag is filled with CHO-S SFM II media and CHO-S cells using sterile tubing and a peristaltic pump to final concentration of 5×10⁵ cells per ml. This is done 1 day before the run is scheduled to start. The platform is set to rock at 15 and the culture and the cell are grown overnight. The following day the cells are counted and viability is determined: the target cell density is 1×10⁶ cells/ml and a viability of 99%. If these criteria are met the wave run is initiated.

The DNA plasmid concentration for the transfection is about 1 mg/L of cell culture volume (or 0.5 mg each of heavy and light chain plasmids). The appropriate volume of stock plasmids is aliquoted into 100 ml of OptiPro media. PEI (3 mg/L final transfection concentration) is aliquoted into 25 ml of OptiPro media. The PEI solution is then added to the plasmid solution and mixed. Complex formation is allowed to proceed for 5 min. The plasmid DNA/PEI solution is then added to the wave bag using the sterile tube and peristaltic pump set-up previously used to load cells and media. The CHO-S cells are then rocked for 4 hours at 37° C. to allow for transfection to proceed.

After the 4 hour incubation the cells are diluted 1:2 with FortiCHO media previously warmed to 37° C. Prior to adding to the wave bag, the FortiCHO media is supplemented with Pen/Strep Amphotericin at 2X the final volume yielding a 1X concentration in the final culture volume. The FortiCHO media is also supplemented with 1X GlutaMax. The supplemented FortiCHO media is then pumped into the bag using the sterile tubing set-up. This effectively dilutes the cell 1:2. The cells are then rocked overnight at 37° C.

After the overnight incubation at 37° C. the temperature of the wave bag is reduced to 28° C. The rocking frequency is increased to 18-20. A sample of cells is obtained and a cell count and viability is determined (trypan blue). The wave run is monitored (cell count, viability, titer) every other day for the duration of the run. The wave run is terminated when the viability of the cells begins to drop below 80%. The run is terminated by pumping the cells and media out of the bag through a ZetaPlus filtration unit and the cell free media filtrate is collected. The clarified cell media is then sterile filtered using a sterilized disposable capsule filter unit (LifeAssure PLA Series, 3M Purifications, Inc.). The filtered media is collected sterilely and held at 4° C. for further processing.

We claim:

1. A recombinant pharmaceutical composition comprising a plurality of different secreted recombinant polypeptides selected from the group consisting of immunoglobulins, Fab fragments, scFv antibodies scFv-Fc antibodies and combinations thereof.

2. The recombinant pharmaceutical composition of claim 1, wherein the differences between the antibodies are the sequences of their variable domain regions in a heavy chain and a light chain.

3. The recombinant pharmaceutical composition of claim 1, wherein the differences between the antibodies are the sequences of their variable domain regions in a heavy chain and a light chain.

4. The recombinant pharmaceutical composition of claim 1, wherein the antibodies are in a form selected from the group consisting of IgG1, IgG2, IgG3, IgG4, IgA1, IgA2, IgM, IgD and IgE and combination thereof.

5. The recombinant pharmaceutical composition of claim 1, wherein the pharmaceutical composition comprises at least about 20 different secreted recombinant polypeptides.

6. The recombinant pharmaceutical composition of claim 1, wherein the pharmaceutical composition comprises at least about 100 different secreted recombinant polypeptides.

7. The recombinant pharmaceutical composition of claim 1, wherein the pharmaceutical composition comprises at least about 1000 different secreted recombinant polypeptides.

8. The recombinant pharmaceutical composition of claim 1, wherein the pharmaceutical composition comprises at least about 10,000 different secreted recombinant polypeptides.

9. A cell based method to manufacture, by recombinant means, fully human polyclonal antibody formulations comprising varying proportions of antibody classes and subclasses.

10. The cell based method to manufacture, by recombinant means, fully human polyclonal antibody formulations of claim 9, wherein the antibody classes are selected from the group consisting of IgG antibodies, Ig antibodies, IgA antibodies, scFv single chain antibodies, Fab antibody fragments, and combinations thereof.

11. A method for treating a disease, comprising administering an effective amount of a recombinant pharmaceutical composition comprising a plural of different secreted recombinant polypeptides selected from the group consisting of immunoglobulins, Fab fragments, scFv antibodies and combinations thereof.

12. A method for treating a disease of claim 11, wherein the recombinant pharmaceutical composition comprising at least 20 different secreted recombinant polypeptides.

13. A method for treating a disease of claim 11, wherein the recombinant pharmaceutical composition comprising at least 100 different secreted recombinant polypeptides.

14. A method for treating a disease of claim 11, wherein the recombinant pharmaceutical composition comprising at least 1000 different secreted recombinant polypeptides.

15. A method for treating a disease of claim 11, wherein the recombinant pharmaceutical composition comprising at least 10⁴ different secreted recombinant polypeptides.

16. A method for treating a disease involving mucosal tissue, lung tissue or eye tissue, comprising administering an effective amount of a recombinant pharmaceutical composition comprising a plurality of different secreted recombinant polypeptides selected from the group consisting of immunoglobulins, Fab fragments, scFv antibodies and combinations thereof.

17. The method for treating a disease involving mucosal tissue, lung tissue or eye tissue of claim 16, wherein the recombinant pharmaceutical composition contains primarily polyclonal IgA class antibodies.

18. The method for treating a disease involving mucosal tissue, lung tissue or eye tissue of claim 16, wherein the pharmaceutical composition comprises at least about 20 different secreted recombinant polypeptides.

19. The method for treating a disease involving mucosal tissue, lung tissue or eye tissue of claim 16, wherein the pharmaceutical composition comprises at least about 100 different secreted recombinant polypeptides.

20. The method for treating a disease involving mucosal tissue, lung tissue or eye tissue of claim 16, wherein the pharmaceutical composition comprises at least about 1000 different secreted recombinant polypeptides.

21. The method for treating a disease involving mucosal tissue, lung tissue or eye tissue of claim 16, wherein the pharmaceutical composition comprises at least about 10,000 different secreted recombinant polypeptides.
